(19) United States
(12) Patent Application Publication
(10) Pub. No.: US 2026/0215841 A1
(43) Pub. Date: Jul. 30, 2026

(54) DIRECTIONAL ABLATION PROBE AND METHODS OF USING THE SAME

(71) Applicant: Medtronic Europe Sàrl, Tolochenaz (CH)

(72) Inventor: Sachin P. Budhabhatti, Unionville, CT (US)

(21) Appl. No.: 19/146,404

(22) PCT Filed: Jan. 15, 2024
(86) PCT No.: PCT/EP2024/050801

§ 371 Date: Jul. 8, 2025

Related U.S. Application Data
(60) Provisional application No. 63/440,854, filed on Jan. 24, 2023.

(51) Int. Cl. A61B 18/14; A61B 18/00

(52) U.S. Cl. CPC A61B 18/148; A61B 2018/00023; A61B 2018/00101; A61B 2018/00577

(57) ABSTRACT

A probe according to at least one embodiment of the present disclosure includes a distal electrode disposed on a distal end of the probe; a proximal electrode disposed proximally from the distal electrode; and a thermal shield disposable on the distal end of the probe to selectively thermally insulate at least one of the distal electrode and the proximal electrode, the thermal shield including an aperture that enables dissipation of thermal energy from at least one of the distal electrode and the proximal electrode through the aperture.

### BACKGROUND

The present disclosure is generally directed to surgical tools, and relates more particularly to surgical tools capable of ablating anatomical tissue.

Various surgical tools are often required to successfully complete surgical procedures. Certain types of tools may be required for different procedures. Some surgical procedures utilize applied electrical current for ablation. Bipolar ablation may occur when current passes through anatomical tissue positioned between two electrodes.

### BRIEF SUMMARY

Example aspects of the present disclosure include:

A probe according to at least one embodiment of the present disclosure comprises: a distal electrode disposed on a distal end of the probe; a proximal electrode disposed proximally from the distal electrode; and a thermal shield disposable on the distal end of the probe to selectively thermally insulate at least one of the distal electrode and the proximal electrode, the thermal shield including an aperture that enables dissipation of thermal energy from at least one of the distal electrode and the proximal electrode through the aperture.

Any of the features herein, wherein a position of the aperture relative to the at least one of the distal electrode and the proximal electrode is adjustable to change an amount of thermal energy dissipated from the probe.

Any of the features herein, wherein the thermal shield is rotatable relative to at least one of the distal electrode and the proximal electrode.

Any of the features herein, wherein the probe further comprises: a thermally insulative element configured to translate across the aperture to adjust an amount of thermal energy dissipated from the probe.

Any of the features herein, wherein the thermal shield comprises a thermally insulative material.

Any of the features herein, wherein the thermally insulative material is at least one of plastic, glass, and polyurethane.

Any of the features herein, wherein the thermal shield comprises a distal end cap connectable to the distal end of the probe.

Any of the features herein, wherein the distal end cap includes a lock that connects the distal end cap to the probe.

Any of the features herein, wherein the lock comprises at least one of a mechanical lock, a fixed lock, a friction lock, and a thermal lock.

Any of the features herein, wherein the aperture is disposed on a distal tip of the distal end cap.

A system according to at least one embodiment of the present disclosure comprises: a probe comprising: a distal electrode disposed on a distal end of the probe; and a proximal electrode disposed proximally from the distal electrode; and a thermal shielding disposable on the probe and configured to selectively thermally insulate at least one of the distal electrode and the proximal electrode, the thermal shielding comprising: an aperture through which thermal energy from at least one of the distal electrode and the proximal electrode can be dissipated.

Any of the features herein, wherein a position of the aperture relative to the at least one of the distal electrode and the proximal electrode is adjustable to change an amount of thermal energy dissipated from the probe.

Any of the features herein, wherein the thermal shielding is rotatable relative to at least one of the distal electrode and the proximal electrode.

Any of the features herein, wherein the probe further comprises: a thermally insulative element configured to translate across the aperture to adjust an amount of thermal energy dissipated from the probe.

Any of the features herein, wherein the thermal shielding comprises a thermally insulative material.

Any of the features herein, wherein the thermally insulative material is at least one of plastic, glass, and polyurethane.

Any of the features herein, wherein the thermal shielding comprises a distal end cap connectable to the distal end of the probe.

Any of the features herein, wherein the distal end cap includes at least one of a mechanical lock, a fixed lock, a friction lock, and a thermal lock that connects the distal end cap to the probe.

Any of the features herein, wherein the aperture is disposed on a distal tip of the distal end cap.

A system according to at least one embodiment of the present disclosure comprises: a cooling apparatus including an input channel and an output channel; a probe comprising: an active electrode disposed on a distal end of the probe; and a return electrode disposed proximally from the active electrode; and a thermal sleeve disposable over the probe to selectively thermally insulate at least one of the active electrode and the return electrode, the thermal sleeve including an aperture that enables dissipation of thermal energy from the active electrode through the aperture.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

Further disclosed herein is a probe that includes a distal electrode disposed on a distal end of the probe; a proximal electrode disposed proximally from the distal electrode; and a thermal shield disposable on the distal end of the probe to selectively thermally insulate at least one of the distal electrode and the proximal electrode, the thermal shield including an aperture that enables dissipation of thermal energy from at least one of the distal electrode and the proximal electrode through the aperture.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1A is a perspective view of aspects of a surgical probe according to at least one embodiment of the present disclosure;

FIG. 1B is a detailed perspective view of a distal end of the surgical probe according to at least one embodiment of the present disclosure;

FIG. 1C is another detailed perspective view of the distal end of the surgical probe according to at least one embodiment of the present disclosure;

FIG. 1D is a cross-sectional plan view of a housing of the surgical probe according to at least one embodiment of the present disclosure;

FIG. 1E is a cross-sectional perspective view of the housing of the surgical probe according to at least one embodiment of the present disclosure;

FIG. 2A is a detailed perspective view of a distal end of a surgical probe with a thermal shield according to at least one embodiment of the present disclosure;

FIG. 2B is an alternative detailed perspective view of the distal end of the surgical probe with the thermal shield according to at least one embodiment of the present disclosure;

FIG. 2C is a plan view of the surgical probe with a closure in a first position according to at least one embodiment of the present disclosure;

FIG. 2D is a plan view of the surgical probe with the closure in a second position according to at least one embodiment of the present disclosure;

FIG. 2E is a plan view of the surgical probe with the closure in a third position according to at least one embodiment of the present disclosure;

FIG. 2F is a plan view of the surgical probe with the closure in a fourth position according to at least one embodiment of the present disclosure;

FIG. 2G is a detailed perspective view of a distal end of a surgical probe with a cap according to at least one embodiment of the present disclosure;

FIG. 3 is a block diagram of a system according to at least one embodiment of the present disclosure; and

FIG. 4 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10× Fusion processors; Apple A11, A12, A12X, A12Z, or A13 Bionic processors; or any other general purpose microprocessors), graphics processing units (e.g., Nvidia Geforce RTX 2000-series processors, Nvidia Geforce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

Ablating anatomical tissue (e.g., bone, tissue, nerves, etc.) can pose a risk to one or more critical structures (e.g., the spinal cord which, if damages, could leave the patient paralyzed) in or around the ablation zone due to the exothermic nature of ablation. To minimize the risk of damaging critical structures, tumors or lesions in or around the critical structures may be left untreated. As a result, a patient with, for example, 50% of the lesions in a given vertebral body near critical structures could only have half the lesions treated, resulting in possible patient dissatisfaction.

The safe treatment of lesions near critical structures may be provided with systems and methods of the present disclosure, which may result in complete lesion care and pain relief for the patient. A direction ablation probe according to at least one embodiment of the present disclosure can be oriented in a desired direction to provide lesion treatment for lesions located near critical structures without damaging the critical structures.

In some embodiments, the direction probe may comprise a thermal shielding disposed on a distal end of the probe that allows heat to propagate along the portion of the probe exposed to anatomical tissue. In some embodiments, the thermal shield may be cooled, such that anatomical tissue proximate the thermal shield remains unablated. Additionally or alternatively, the probe may comprise a thermally insulated sheath or cover that includes one or more cutouts, apertures, or slots through which heat generated by the probe can be dissipated. The sheath or cover may be rotatable relative to the probe, such that anatomical tissues in different locations can be ablated.

Embodiments of the present disclosure provide technical solutions to one or more of the problems of (1) ablating lesions or tumors near critical structures and (2) charring of anatomical tissues when performing ablations.

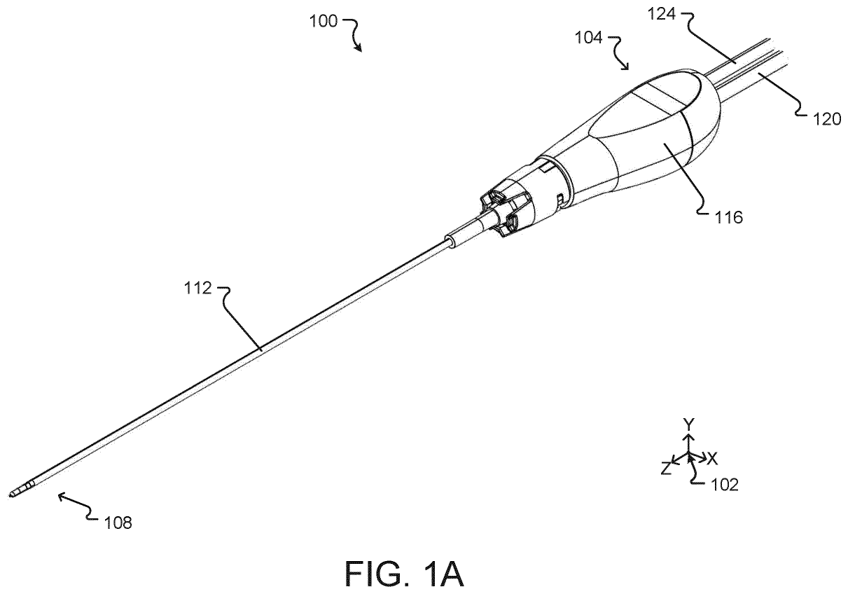

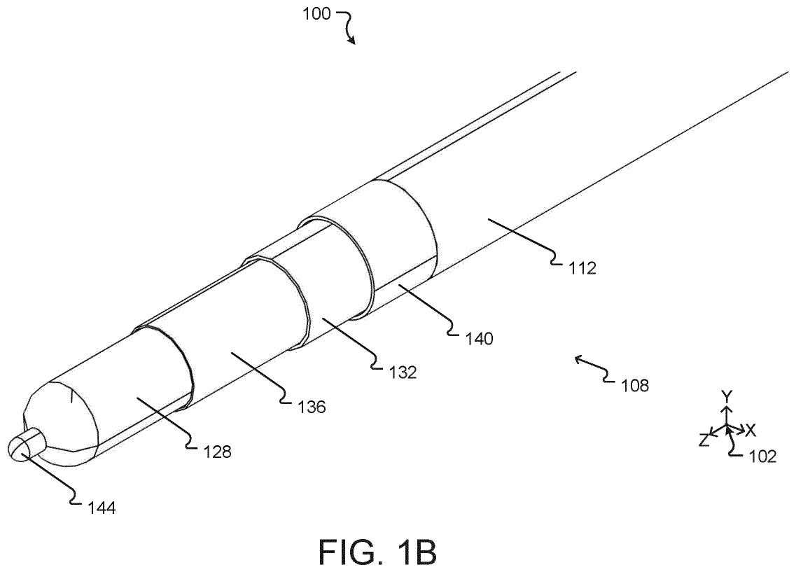

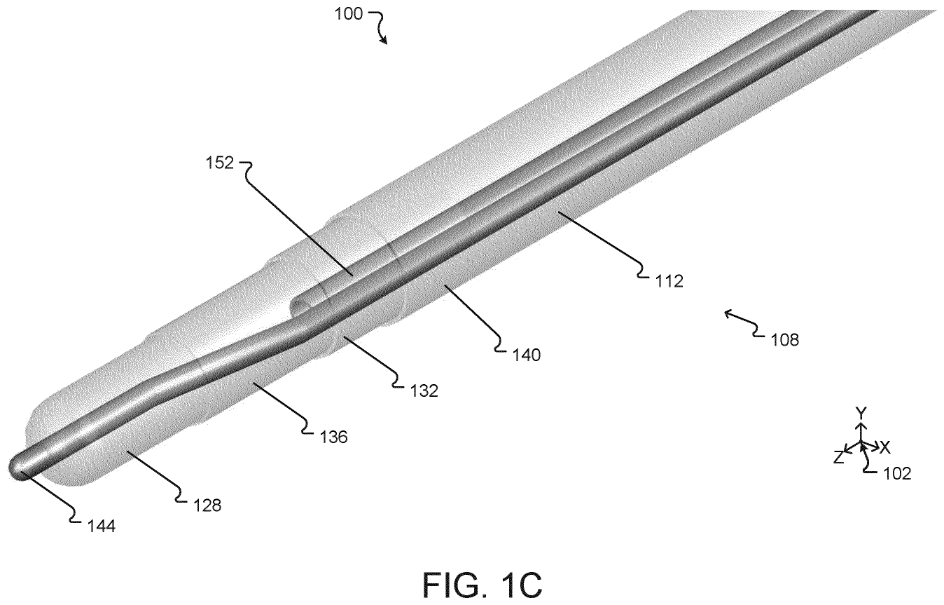

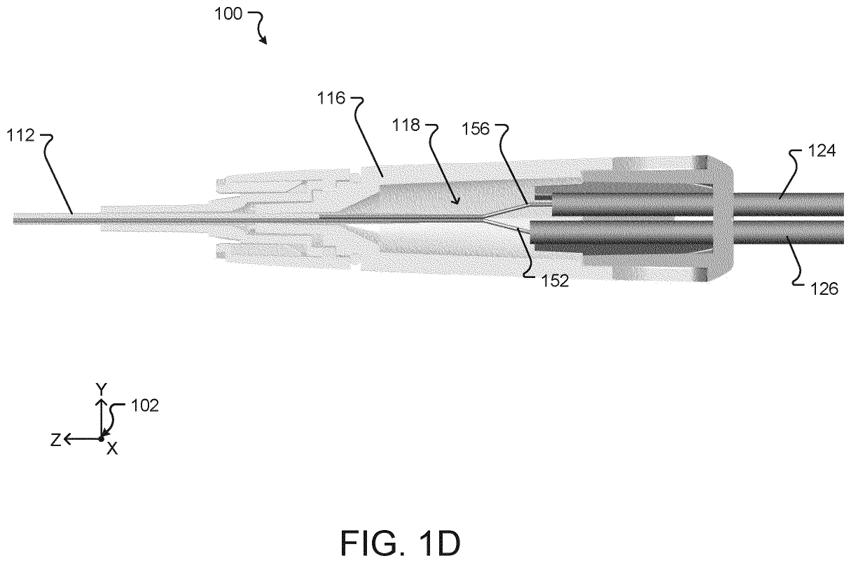

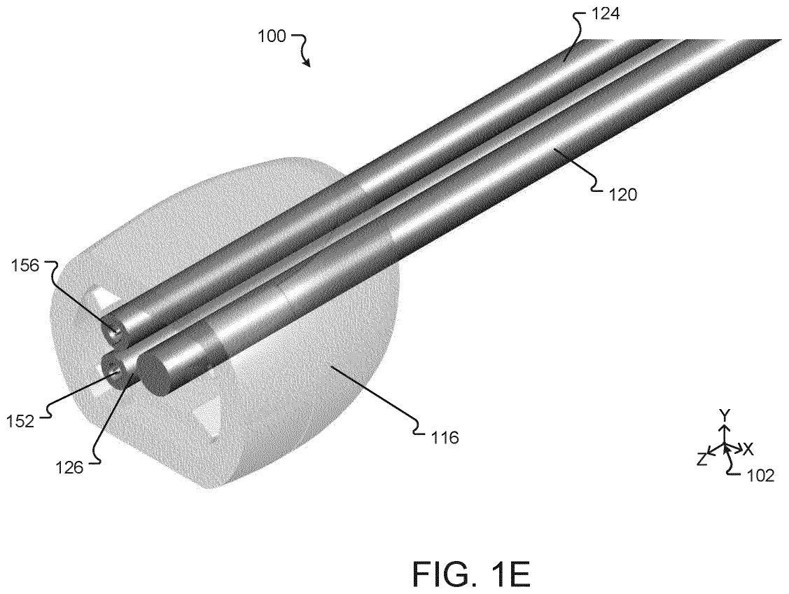

Turning first to FIGS. 1A-1E, aspects of a probe 100 according to at least one embodiment of the present disclosure are shown. The probe 100 may be used to perform ablation of anatomical tissue and/or to carry out one or more other aspects of one or more of the methods disclosed herein. The probe 100 extends from a proximal end 104 to a distal end 108 and includes an elongated shaft 112, a clamshell 116, an electric cable 120, a coolant output conduit 124, and a coolant input conduit 126.

In some embodiments, reference may be made to dimensions, angles, directions, relative positions, and/or movements associated with one or more components of the probe 100 with respect to a coordinate system 102. The coordinate system 102, as shown in the accompanying figures, includes three-dimensions comprising an X-axis, a Y-axis, and a Z-axis. Additionally or alternatively, the coordinate system 102 may be used to define planes (e.g., the XY-plane, the XZ-plane, and the YZ-plane) of the probe 100. These planes may be disposed orthogonal, or at 90 degrees, to one another. While the origin of the coordinate system 102 may be placed at any point on or near the probe 100, for the purposes of description, the axes of the coordinate system 102 are disposed along the same directions from figure to figure. Additionally or alternatively, the directionality of the X-axis, Y-axis, and Z-axis may be flipped, as noted with negative directionality (e.g., the negative X-axis direction is the opposite direction of the X-axis direction illustrated by the direction of the associated arrow).

The clamshell 116 may provide a location for the physician to grip the probe 100 when inserting or extracted the probe 100 to or from a surgical site. The clamshell 116 may also provide a housing for the elongated shaft 112, the electric cable 120, the coolant output conduit 124, and/or the coolant input conduit 126, and/or for any of the other components of the probe 100 (or portions thereof). As illustrated in FIGS. 1D and 1E, the clamshell 116 may provide an interior cavity 118 through which the electrical cable 120 may extend. The electric cable 120 may be or comprise an insulative tubing containing the current-carrying wiring. The current-carrying wiring connects to a first electrode 128 and a second electrode 132, forming a circuit through which current can pass. The wiring may carry current generated by a generator or other power source (discussed below) into the probe 100. In some embodiments, the clamshell 116 may encompass portions of electrodes of the probe 100 to which the electric cable 120 is connected. In such embodiments, the current-carrying wiring may be connected to both the electrodes in the clamshell 116, such that current can flow into the first electrode 128 from an active wire (e.g., a wire carrying current connected to a generator), through anatomical tissue proximate the probe 100, into the second electrode 132, and from the second electrode 132 back out of the probe through a return wire (e.g., a wire that carries current back to the generator).

The electric cable 120 may also carry or pass a thermocouple 144 into the probe 100. The thermocouple 144 may be or comprise one or more sensors or thermally sensitive material that measure the temperature of the electrodes of the probe 100. In some embodiments, each electrode of the probe 100 may be monitored by a separate thermocouple. The thermocouple 144 may, based on the heating experienced by the electrodes 128, 132 during operation, generate one or more measurements that represent the temperature of the electrodes 128, 132. In some embodiments, the thermocouple 144 may extend from the electric cable 120 into the clamshell 116, and may be threaded through or otherwise be disposed in the interior of the probe 100. As illustrated in FIGS. 1C and 1E, the thermocouple 144 may run along the length of the probe 100 from the proximal end 104 into the distal end 108 to measure the temperature of the electrodes. In some embodiments, the thermocouple 144 may be or comprise a type K thermocouple (e.g., a thermocouple with a temperature range of about 0 degrees Celsius (° C.) to about 1260° C.), a type J thermocouple (e.g., a thermocouple with a temperature range of about 0° C. to about 760° C.), a type N thermocouple (e.g., a thermocouple with a temperature range of about 0° C. to about 1260° C.), a type B thermocouple (e.g., a thermocouple with a temperature range of about 870° C. to about 1700° C.), a type E thermocouple (e.g., a thermocouple with a temperature range of about 0° C. to about 870° C.), a type R thermocouple (e.g., a thermocouple with a temperature range of about 500° C. to about 1500° C.), a type S thermocouple (e.g., a thermocouple with a temperature range of about 500° C. to about 1500° C.), or a type C thermocouple (e.g., a thermocouple with a temperature range of about 0° C. to about 2300° C.). The different types of thermocouples listed above may have different compositions of materials, such that the thermocouple can function at the specified temperature range while capturing temperature measurements. In some embodiments, the type of thermocouple may be selected based on the type of surgery, the anticipated temperature of the electrodes, the anticipated amount of time needed to ablate anatomical tissue, the surgical plan, combinations thereof, and the like. In some embodiments, the probe 100 may comprise multiple thermocouples to measure the temperature of one or more components (e.g., the electrodes of the probe 100).

The coolant input conduit 126 and the coolant output conduit 124 may comprise a coolant input channel 152 and a coolant output channel 156, respectively. The coolant input channel 152 may be or comprise a hollow, waterproof tube that carries fresh coolant (e.g., water, saline, etc.) into the probe 100 to cool (e.g., absorb heat from) the first electrode 128 and/or the second electrode 132. The coolant output channel 156 may be or comprise a hollow, waterproof tube that transfers spent coolant (e.g., coolant that has absorbed heat from the first electrode 128 and/or the second electrode 132) out of the probe 100. In some embodiments, the coolant input channel 152 and/or the coolant output channel 156 may extend through the elongated shaft 112 and into the distal end 108 of the probe 100. In other embodiments, the coolant input channel 152 may extend into the distal end 108 of the probe 100, while the coolant output channel 156 extends partially into the elongated shaft 112. In such embodiments, the coolant output channel 156 may exert a negative pressure and/or suction force to extract the coolant dispensed by the coolant input channel 152 in the distal end 108 of the probe 100 after the coolant has cooled the first electrode 128 and/or the second electrode 132.

FIG. 1B illustrates a detailed perspective view of the distal end 108 of the probe 100. The distal end 108 may include the first electrode 128, the second electrode 132, an insulation layer 136, a protective barrier 140, and the thermocouple 144.

The first electrode 128 and the second electrode 132 may conduct electrical current (e.g., an RF current) through proximal anatomical tissue thereto in order to ablate the anatomical tissue. The first electrode 128 may be, for example, an active electrode configured to receive current from current-carrying wire and carry the current to the surgical site at the distal end 108 of the probe 100. The second electrode 132 may be, for example, a return electrode configured to receive the current after the current has passed through the anatomical tissue at the surgical site and carry the current back through wiring to the generator or other power source to complete the circuit. In some embodiments, the first electrode 128 and/or the second electrode 132 may be or comprise hollow cylindrical tubes (e.g., stainless steel tubing, spring steel tubing, tubing comprising other metal alloys, etc.) extending from the proximal end 104 of the probe 100 (e.g., from within the interior cavity 118 of the clamshell 116) toward the distal end 108 of the probe 100. The first electrode 128 and/or the second electrode 132 may extend through a hollow interior of the elongated shaft 112. In some embodiments, the first electrode 128 may extend further toward the distal end 108 of the probe 100 than the second electrode 132.

In some embodiments, an insulation layer 136 may be disposed at least partially around the first electrode 128 or, more generally, between the first electrode 128 and the second electrode 132. The insulation layer 136 may be or comprise insulative material (e.g., plastic, PVC, Teflon, rubber, combinations thereof, etc.) capable of preventing current from passing from the first electrode 128 and/or the second electrode 132 to other components of the probe 100. Additionally or alternatively, the insulation layer 136 may electrically separate the first electrode 128 and the second electrode 132, such that current passes through anatomical tissue when travelling from the first electrode 128 to the second electrode 132 (or vice versa).

The protective barrier 140 may be or comprise insulative material, such as Polyimide (PI) material that protects the second electrode 132 (e.g., prevents patient anatomy or other anatomical tissues from contacting the second electrode 132). The protective barrier 140 may be disposed at least partially around the second electrode 132 or, more generally, between the second electrode 132 and the elongated shaft 112. The protective barrier 140 may cover the portion of the second electrode 132 disposed outside the clamshell 116, which may beneficially increase safety and provide an insulative layer that can prevent or mitigate electric shock in the event the physician contact the second electrode 132 when the generator or power source is on.

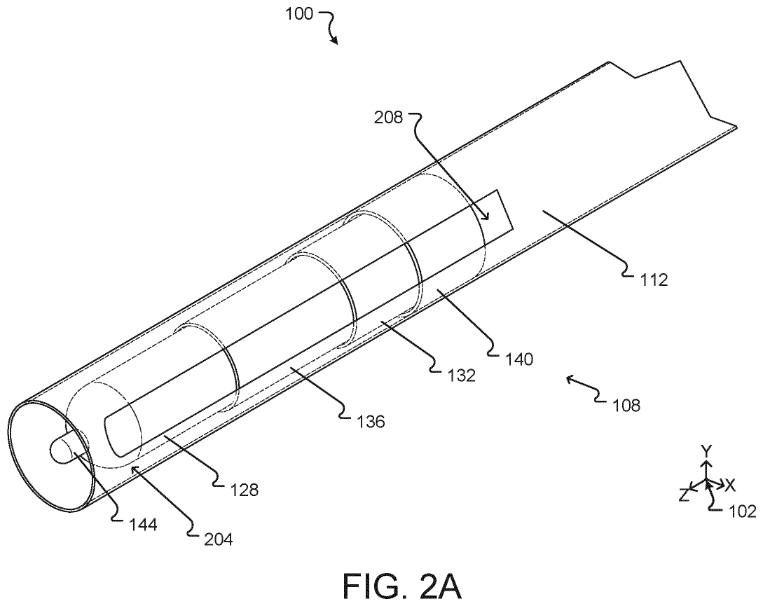

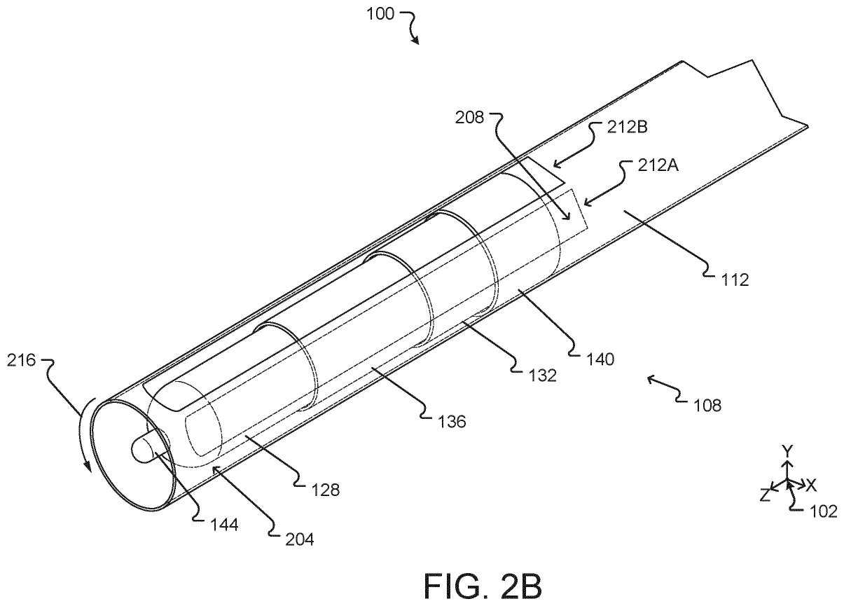

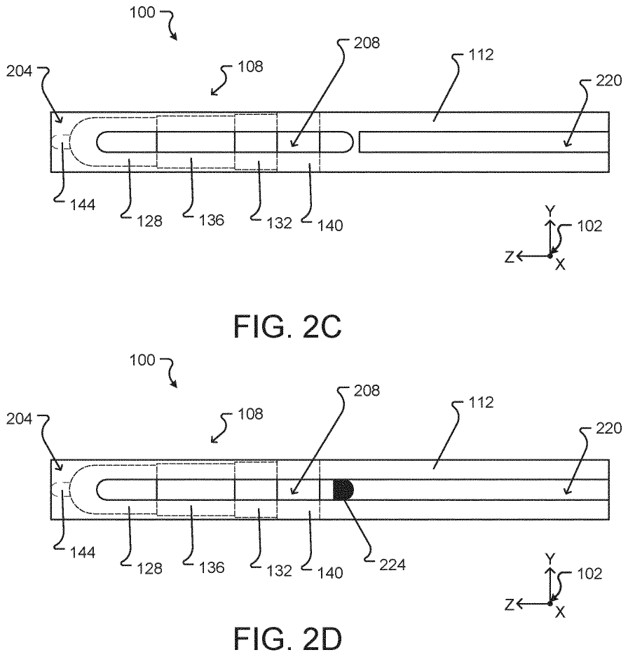

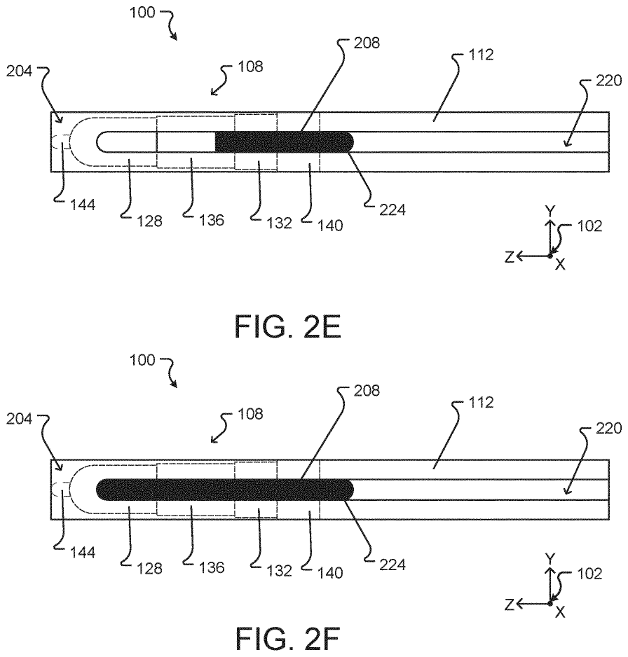

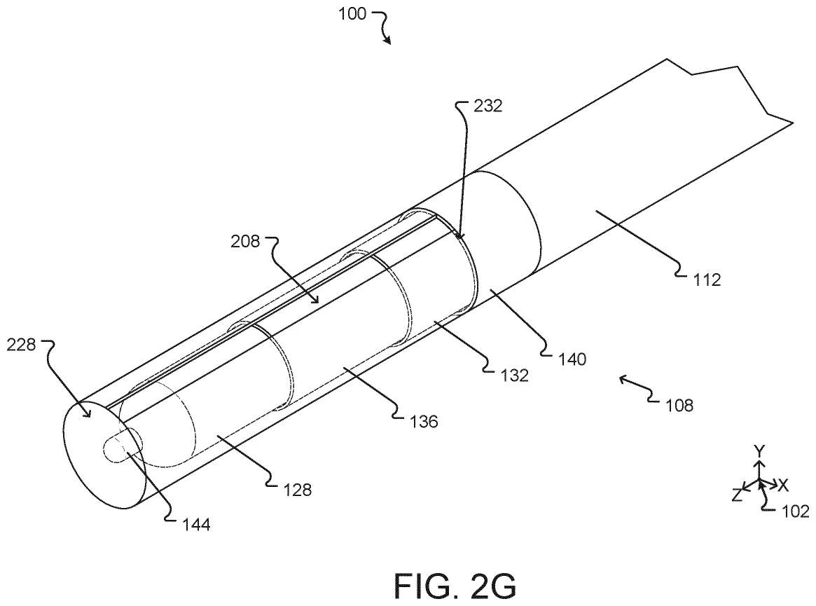

FIGS. 2A-2G illustrate additional aspects of the probe 100 in accordance with embodiments of the present disclosure. The probe 100 comprises a thermal shield 204 disposable on the distal end 108 of the probe 100, as depicted in FIGS. 2A and 2B. The thermal shield 204 comprises an aperture 208 and a closure 220.

The thermal shield 204 is an insulative element (e.g., a thermal sleeve) capable of being slid over, wrapped around, connected to, or otherwise disposed on the distal end 108 of the probe 100 to thermally insulate the probe 100 from the surrounding environment (e.g., surrounding anatomical tissue, which may include critical structures such as spinal cord tissue). In some embodiments, the thermal shield 204 may be or comprise a thermal sleeve that can be slid over the end of the distal end 108. The thermal shield 204 extends from the distal end 108 of the probe 100 to the proximal end 104 of the probe 100, such that a user of the probe 100 (e.g., a physician) can access, adjust, or otherwise interact with the thermal shield 204 when the probe 100 is inserted into the surgical site. In some embodiments, an interior cavity of the thermal shield 204 may be formed to be flush with one or more components of the probe 100. For example, the thermal shield 204 may include a cavity into which the first electrode 128 can be disposed, such that one or more portions of the outer surface of the first electrode 128 is surrounded by the thermal shield 204. The thermal shield 204 may be or comprise one or more thermally insulative materials, such that heat generated by the probe 100 remains in the interior of the thermal shield 204 and is not absorbed by anatomical tissues near the thermal shield 204. Non-limiting examples of the thermally insulative material include Polyimide material, polyurethane, plastic, glass, combinations thereof, and the like.

The aperture 208 of the thermal shield 204 is a hole, opening, or the like disposed in the thermal shield 204 that exposes one or more components of the probe 100 to the surrounding environment (e.g., anatomical tissue). The aperture 208 provides a location from which heat generated by the probe 100 can propagate, dissipate, or otherwise escape from the thermal shield 204. In other words, the thermal shield 204 may block or prevent heat generated by the current flow between the first electrode 128 and the second electrode 132 from escaping in any direction except through the aperture 208. As a result, anatomical tissue positioned along the direction of heat propagation from the aperture 208 may be ablated, while other anatomical tissues proximate the probe 100 avoid ablation. In some embodiments the position of the thermal shield 204 can be adjusted relative to the probe 100, so the aperture 208 can be positioned to selectively ablate anatomical tissue proximate the probe 100. For example, a user may position the probe 100 next to anatomical tissue targeted for ablation (e.g., a tumor or lesion) as well as next to critical structures (e.g., a patient's spinal cord) that should not be ablated, with the aperture 208 positioned such that heat is dissipated in the direction of the target anatomical tissue and away from the critical structures. In this way, the user can ablate the target anatomical tissue while reducing the likelihood of damage to the critical structures.

While the aperture 208 is illustrated as extending along the Z-axis direction of the probe 100, it is to be understood that the aperture 208 may be positioned anywhere on the thermal shield 204. For example, the aperture 208 may be disposed on a distal tip of the thermal shield 204, such that heat generated by the probe 100 is dissipated from the distal tip of the probe 100 along the Z-axis direction. In some embodiments, the aperture 208 may comprise a plurality of smaller apertures disposed at various locations on the thermal shield 204, such that various amounts of heat can be dissipated in different directions. As an example, the aperture 208 may comprise two separate apertures extending along the Z-axis direction such that heat generated by the probe 100 can be dissipated in the X-axis direction and the negative X-axis direction. Additionally or alternatively, the aperture 208 may be or comprise one or more different shapes depending on, for example, the type of probe, the type of surgery or surgical procedure, characteristics of the anatomical tissue subject to ablation, combinations thereof, and the like. The shape of the aperture 208 is in no way limited, and the aperture 208 may be circular (e.g., formed in the shape of a circle, an oval, an ellipse, etc.), rectilinear, rectangular, trapezoidal, or the like. The various shapes of the aperture 208 may enable a variety of heat gradients and/or ablation zones to be formed during ablation, which may beneficially enable a physician to select different heat shields with different apertures to address various surgical situations.

In some embodiments and as previously mentioned, the position of the thermal shield 204 may be rotatable, translatable, or otherwise adjustable relative to the probe 100. As shown in FIG. 2B, the thermal shield 204 may configured to rotate with respect to the probe 100 (e.g., in a clockwise direction, in a counterclockwise direction, etc.). The rotation of the thermal shield 204 relative to the probe 100 may move the aperture 208, which subsequently adjusts the direction in which heat generated by the probe 100 is dissipated. As depicted in FIG. 2B, the thermal shield 204 may rotate relative to the probe 100 in a direction indicated by arrow 216, such that the aperture 208 moves from a first position 212A to a second position 212B. When in the first position 212A, heat generated by the probe 100 may be dissipated in the X-axis direction. After the thermal shield 204 has rotated, the aperture 208 may be in the second position 212B, such that heat generated by the probe 100 can be dissipated in the Y-axis direction. While the movement of the thermal shield 204 in FIG. 2B is depicted as rotational, it is to be understood that the thermal shield 204 may be moveable in three-dimensional (3D) space relative to the probe 100. For example, the thermal shield 204 may additionally or alternatively be configured to translate relative to the probe 100 (e.g., along the Z-axis direction, along the negative Z-axis direction, etc.). The thermal shield 204 may include one or more locking mechanisms (not shown) such that, once the thermal shield 204 is adjusted or otherwise moved relative to the probe 100, the thermal shield 204 can be locked in place to fix the location of the aperture 208 and the resultant direction in which heat is dissipated.

The closure 220 is a thermally insulative element configured to slide, translate, or otherwise move across the aperture 208 to adjust the amount and/or location of heat dissipated from the probe 100. The closure 220 may be disposed on an inner surface of the thermal shield 204 (e.g., mounted on a railing system disposed on the inner surface of the thermal shield 204) and extend from the thermal shield 204 to the proximal end 104 of the probe 100. The user of the probe 100 can adjust the position of the closure 220 relative to the thermal shield 204 to selectively open or close the aperture 208 or a portion thereof. As shown in FIGS. 2C-2F, the closure 220 may be moved along the Z-axis direction to adjust a surface area of the probe 100 exposed to the anatomical tissue proximate the aperture 208. In other words, as the closure 220 is moved along the Z-axis direction, the amount of heat dissipated by the aperture 208 may change. As shown in FIG. 2C, the closure 220 can be positioned such that the aperture 208 is not blocked. The user may adjust the position of the closure 220 (e.g., by pushing a portion of the closure 220 located at the proximal end 104 of the probe 100 in the Z-axis direction), such that the closure 220 slides across the aperture 208. As a result, and as depicted in FIG. 2D, a portion 224 of the aperture 208 is blocked by the closure 220. The blocking of the portion 224 may result in less heat being dissipated from the aperture 208.

In some embodiments, the closure 220 may be moved by the user such that one electrode is blocked from the surgical site, while the other electrode is exposed to the surgical site. In this case, heat from the electrode exposed to the surgical site may escape the thermal shield 204 at a greater rate than heat from the electrode that is blocked by the closure 220. As depicted in FIG. 2E for example, the closure 220 may be moved such that the first electrode 128 is exposed to the surgical site, but the second electrode 132 is not. In some embodiments, the closure 220 may be translatable to block the entirety of the aperture 208 as shown in FIG. 2F, such that the thermal shield 204 completely insulates the probe 100. The closure 220 may absorb or insulate the heat previously dissipated through the aperture 208, such that the anatomical tissue proximate the aperture 208 does not receive the heat and, in some cases, is not ablated.

Turning to FIG. 2G, the thermal shield 204 may be or comprise a cap 228 that is connectable, attachable, or otherwise couplable to the distal end 108 of the probe 100 via a lock 232. In some embodiments, the cap 228 may be or comprise an end cap, such as a distal end cap connectable to the distal end 108 of the probe 100 such that a tip of the probe 100, the first electrode 128, and/or the second electrode 132 is covered by the distal end cap. The cap 228 may be or comprise thermally insulative material (e.g., glass, plastic, polyurethane, etc.) that thermally insulates the probe 100 from the surrounding environment. In some embodiments, the cap 228 may comprise the aperture 208, which may provide a location for heat generated by the probe 100 to propagate or dissipate. In one embodiment, the aperture 208 may be disposed on the distal end of the cap 228. In this case, the cap 228 may be a cylindrical component with the aperture 208 disposed on a first end of the cap 228, such that heat generated by the probe 100 is dissipated along the Z-axis direction.

The lock 232 attaches the cap 228 to the distal end 108 of the probe 100, such that rotational and/or translational movement of the cap 228 is fixed relative to the probe 100. In some embodiments, the cap 228 may be disposed on the distal end 108 in a predetermined position such that, when the physician inserts the probe 100 into the surgical site, the aperture 208 is aligned with the anatomical tissue targeted for ablation (e.g., a lesion or a tumor). The lock 232 may be or comprise a mechanical lock, a fixed lock, a friction lock, a thermal lock, combinations thereof, and the like. The mechanical lock may comprise mechanical components (e.g., locking ball joints, hinges, latches, springs, etc.) that mechanically couple the cap 228 to one or more portions of the probe 100 (e.g., to the second electrode 132, to the elongated shaft 112, etc.). In some embodiments, the probe 100 may comprise a mechanical interface, such that the lock 232 can be coupled with the mechanical interface to attach the cap 228 to the probe 100.

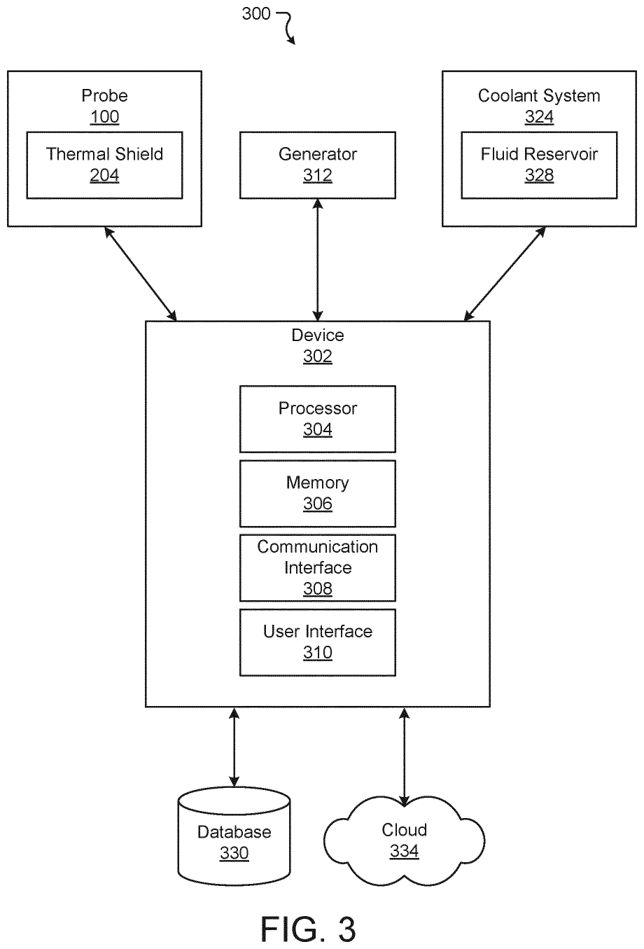

FIG. 3 depicts a block diagram of a system 300 according to at least one embodiment of the present disclosure. The system 300 may be used to control ablation of anatomical tissue using one or more probes, enable user interaction and control of the one or more probes, and/or to carry out one or more aspects of one or more of the methods disclosed herein. The system 300 comprises the probe 100, a device 302, a generator 312, a coolant system 324, a database 330, and a cloud or other network 334. In some embodiments, the system 300 may comprise additional or alternative components to those depicted in FIG. 3.

The device 302 comprises a processor 304, a memory 306, a communication interface 308, and a user interface 310. In some embodiments, the device 302 may comprise more or fewer components than those depicted in FIG. 3.

The processor 304 of the device 302 may be any processor described herein or any similar processor. The processor 304 may be configured to execute instructions stored in the memory 306, which instructions may cause the processor 304 to carry out one or more computing steps utilizing or based on data received from probe 100, the generator 312, the coolant system 324, the database 330, and/or the cloud 334.

The memory 306 may be or comprise RAM, DRAM, SDRAM, other solid-state memory, any memory described herein, or any other tangible, non-transitory memory for storing computer-readable data and/or instructions. The memory 306 may store information or data useful for completing, for example, any step of the methods described herein, or of any other methods. The memory 306 may store, for example, instructions that support one or more functions of the probe 100. For instance, the memory 306 may store content (e.g., instructions) that, when executed by the processor 304, enable cooling of and/or ablation using the probe 100. Such content, if provided as in instruction, may, in some embodiments, be organized into one or more applications, modules, packages, layers, or engines. Alternatively or additionally, the memory 306 may store other types of content or data that can be processed by the processor 304 to carry out the various methods and features described herein. Thus, although various contents of memory 306 may be described as instructions, it should be appreciated that functionality described herein can be achieved through use of instructions, algorithms, data, and/or the like. The data, algorithms, and/or instructions may cause the processor 304 to manipulate data stored in the memory 306 and/or received from or via the probe 100, the generator 312, the coolant system 324, the database 330, and/or the cloud 334.

The communication interface 308 may be used for receiving data or other information from an external source (such as the generator 312, the coolant system 324, the database 330, the cloud 334, and/or any other system or component not part of the system 300), and/or for transmitting instructions or other information to an external system or device (e.g., another device 302, the generator 312, the coolant system 324, the database 330, the cloud 334, and/or any other system or component not part of the system 300). The communication interface 308 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 308 may be useful for enabling the device 302 to communicate with one or more other processors 304 or devices 302, whether to reduce the time needed to accomplish a computing-intensive task or for any other reason.

The user interface 310 may be or comprise a keyboard, mouse, trackball, monitor, television, screen, touchscreen, and/or any other device for receiving information from a user and/or for providing information to a user. The user interface 310 may be used, for example, to receive a user selection or other user input regarding any step of any method described herein. Notwithstanding the foregoing, any required input for any step of any method described herein may be generated automatically by the system 300 (e.g., by the processor 304 or another component of the system 300) or received by the system 300 from a source external to the system 300. In some embodiments, the user interface 310 may be useful to allow a surgeon or other user to modify instructions to be executed by the processor 304 according to one or more embodiments of the present disclosure, and/or to modify or adjust a setting of other information displayed on the user interface 310 or corresponding thereto.

Although the user interface 310 is shown as part of the device 302, in some embodiments, the device 302 may utilize a user interface 310 that is housed separately from one or more remaining components of the device 302. In some embodiments, the user interface 310 may be located proximate one or more other components of the device 302, while in other embodiments, the user interface 310 may be located remotely from one or more other components of the device 302.

The generator 312 may generate current (e.g., RF current, an RF alternating current, etc.) that is passed into the probe 100. The generator 312 may include one or more electrical components (e.g., batteries, resistors, capacitors, inductors, etc.) that facilitate the generation or modulation of current carried to the probe 100. In some embodiments, the generator 312 may generate a fixed current, while in other embodiments the current generated by the generator 312 may be variable and/or capable of being modulated. In some embodiments, a user may be able to control characteristics of the current (e.g., amplitude, frequency, etc.) output by the generator 312. In some embodiments, the device 302 may control the generator 312 based on inputs from the user (e.g., via the user interface 310), instructions stored in the database 330, or the like.

The coolant system 324 may control the cooling of the probe 100, and includes a fluid reservoir 328. The fluid reservoir 328 may have one or more containers that house one or more coolants (e.g., water, saline, etc.). One or more pumps may be controlled to pump the coolant into and out of the probe 100. The coolant may be fluidically communicated to the probe 100 through one or more fluid conduits (e.g., through the coolant input channel 152 and the coolant output channel 156). In some embodiments, the coolant system 324 may comprise a suction mechanism that can be turned on (e.g., begin generating a vacuum to remove coolant from the probe 100) when coolant is supplied to the probe 100. As such, coolant may be dispensed into a distal end of the probe 100 such as by the coolant input channel 152, and may be removed from the distal end by the coolant output channel 156. In one embodiment, the coolant system 324 may cause the coolant to be pumped into the probe 100 to cool the probe 100. The coolant may then be pumped back into a separate container in the fluid reservoir 328. In some embodiments, the coolant system 324 may be controlled by the device 302 and/or by input commands by the user (e.g., via the user interface 310). Additionally or alternatively, the coolant system 324 may be connected to one or more portions of the thermal shield 204 to cool the thermal shield 204. In this case, the coolant system 324 may have input and output channels disposed along the surface and/or within an interior of the thermal shield 204. The coolant system 324 may pump fresh coolant into the thermal shield 204 through the input channel and pump spent coolant out of the thermal shield 204 through the output channel. As a result, the surface of the thermal shield 204 may be cooled, such that the thermal shield 204 and/or anatomical tissue proximate the thermal shield 204 remain at an appropriate temperature when the probe 100 is being used to perform ablation. In some embodiments, the coolant system 324 may be part of a cooling apparatus that can be used to cool the probe 100. The cooling apparatus may comprise the coolant system 324 and/or components thereof, the fluid reservoir 328 and/or components thereof, the coolant output conduit 124, the coolant input conduit 126, the coolant input channel 152, and/or the coolant output channel 156. In some embodiments, the cooling apparatus may comprise additional or alternative components.

The database 330 may store information related to one or more surgical plans (e.g., information related to the type of tissue to be ablated, the position and orientation of one or more anatomical elements of a patient, etc.); information related to the probe 100 (e.g., a model type, a recommended operating temperature or power range, etc.); and/or any other useful information. The database 330 may be configured to provide any such information to the device 302 or to any other device of the system 300 (e.g., to the generator 312, to the coolant system 324, etc.) or external to the system 300, whether directly or via the cloud 334. In some embodiments, the database 330 may be or comprise part of a hospital image storage system, such as a picture archiving and communication system (PACS), a health information system (HIS), and/or another system for collecting, storing, managing, and/or transmitting electronic medical records including image data.

The cloud 334 may be or represent the Internet or any other wide area network. The device 302 may be connected to the cloud 334 via the communication interface 308, using a wired connection, a wireless connection, or both. In some embodiments, the device 302 may communicate with the database 330 and/or an external device via the cloud 334.

The system 300 or similar systems may be used, for example, to carry out one or more aspects of any of the methods described herein. The system 300 or similar systems may also be used for other purposes.

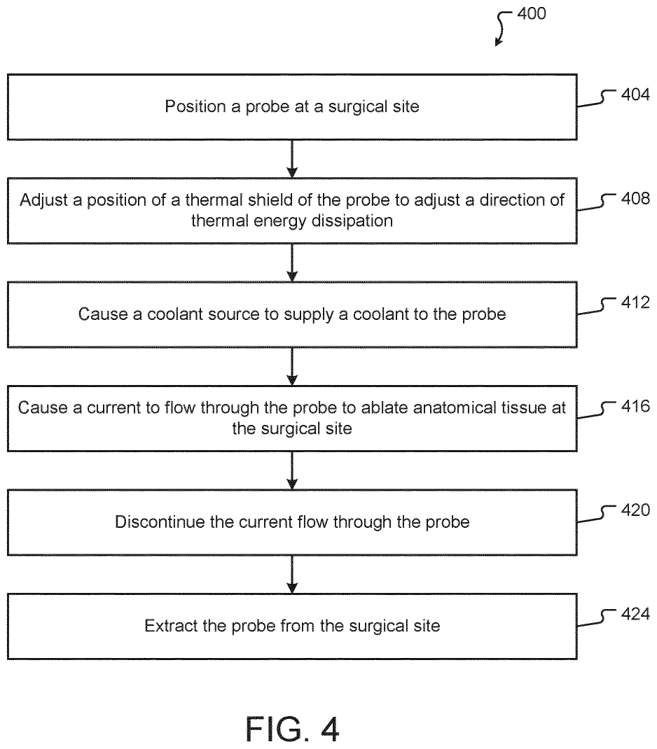

FIG. 4 depicts a method 400 that may be used, for example, to ablate anatomical tissue.

The method 400 comprises positioning a probe at a surgical site (step 404). In some embodiments, the probe may be similar to or the same as the probe 100. The surgical site may be, for example, a vertebra with one or more anatomical elements (e.g., tumors or legions) that are to be ablated by the probe. In some embodiments, the probe may be surgical inserted (e.g., by drilling pedicle tracks and inserting the probes into the vertebra through the tracks). The probe may comprise a distal electrode and a proximal electrode, which may be respectively similar to or the same as the first electrode 128 and the second electrode 132.

The method 400 also comprises adjusting a position of a thermal shield of the probe to adjust a direction of thermal energy dissipation (step 408). The thermal shield may be similar to or the same as the thermal shield 204, and a physician or other user (e.g., a member of surgical staff) may adjust the thermal shield by rotating and/or translating the thermal shield relative to the probe 100. The adjustment of the thermal shield may enable the physician to ablate a first anatomical element (e.g., a lesion) while reducing the likelihood of damaging other anatomical elements (e.g., a portion of the patient's spinal cord). In some embodiments, the thermal shield may be adjusted based on a surgical plan. For example, preoperative and/or intraoperative images may be used to identify locations of critical structures, and the thermal shield may be moved such that heat is dissipated through the aperture of the thermal shield away from any of the critical structures. Once the thermal shield has been adjusted, one or more locking mechanisms may be used by the physician to secure the thermal shield relative to the probe. The securing of the thermal shield may prevent the direction of thermal energy dissipation from changing when the probe is conducting current.

The method 400 also comprises causing a coolant source to supply a coolant to the probe (step 412). In some embodiments, the coolant source may be or comprise a coolant system similar to or the same as the coolant system 324. The coolant source may pump coolant into the probe while the probe performs ablation to cool one or more electrodes of the probe. In some embodiments, the coolant may flow from a fluid reservoir (e.g., fluid reservoir 328) and into the probe via fluid conduits. The spent coolant may then flow back out of the second probe and into a container within the fluid reservoir. In some embodiments, the coolant source may be operatively connected to the thermal shield, such that the outer surface of the thermal shield in contact with non-ablated anatomical tissue (e.g., critical structures such as the spinal cord) remains cooled when the probe is conducting current.

The method 400 also comprises causing a current to flow through the probe to ablate anatomical tissue at the surgical site (step 416). The current may be generated using, for example, a generator such as the generator 312 or any other generator. The current generated may be sufficient to ablate the anatomical tissue in the surgical site. In some embodiments, the thermal shield on the probe may be adjusted as current is flowing, such that heat dissipated from the probe ablates different anatomical tissues proximate the probe. In some embodiments, a closure may be used by the physician to adjust an amount of thermal energy being dissipated from the probe. For example, the physician may determine (e.g., via thermocouple measurements) that the anatomical tissue is receiving too much thermal energy from the probe, and may maneuver the closure such that a portion of the aperture is blocked. As a result, less heat may propagate into the anatomical tissue, lowering the temperature and reducing the likelihood of charring. Similarly, the ablation may begin with the closure blocking a portion of the aperture, and the physician may determine that the anatomical tissue requires additional thermal energy to achieve ablation. In this case, the physician may slide the closure away from the aperture, such that a greater amount of thermal energy is dissipated into the anatomical tissue.

The method 400 also comprises discontinuing the current flow through the probe (step 420). Once it is determined that the target anatomical tissue has been ablated, the current flow through the probe may be discontinued. For example, the physician and/or a control device (e.g., device 302) may determine that ablation is complete (e.g., based on thermocouple measurements, based on the anticipated timing of ablation, etc.), and may disable the generator or instruct the generator to discontinue supplying current to the probe.

The method 400 also comprises extracting the probe from the surgical site (step 424). The probe may be extracted from the surgical site once ablation has been performed to the surgical site. In some embodiments, the method 400 (or one or more steps thereof) may be repeated for one or more target anatomical tissues or elements at the surgical site. For example, in cases where ablation is to be performed on a plurality of lesions or tumors, the probe may remain in the surgical site, and the physician may adjust the thermal shield such that the thermal energy is propagated into another lesion or tumor.

The present disclosure encompasses embodiments of the method 400 that comprise more or fewer steps than those described above, and/or one or more steps that are different than the steps described above.

As noted above, the present disclosure encompasses methods with fewer than all of the steps identified in FIG. 4 (and the corresponding description of the method 400), as well as methods that include additional steps beyond those identified in FIG. 4 (and the corresponding description of the method 400). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

Further disclosed herein is the subject-matter of the following clauses:

1. A probe, comprising:
a distal electrode disposed on a distal end of the probe;
a proximal electrode disposed proximally from the distal electrode; and
a thermal shield disposable on the distal end of the probe to selectively thermally insulate at least one of the distal electrode and the proximal electrode, the thermal shield including an aperture that enables dissipation of thermal energy from at least one of the distal electrode and the proximal electrode through the aperture.

2. The probe of clause 1, wherein a position of the aperture relative to the at least one of the distal electrode and the proximal electrode is adjustable to change an amount of thermal energy dissipated from the probe.

3 The probe of clause 2, wherein the thermal shield is rotatable relative to at least one of the distal electrode and the proximal electrode.

4. The probe of clause 1 or of any of the preceding clauses, wherein the probe further comprises:
a thermally insulative element configured to translate across the aperture to adjust an amount of thermal energy dissipated from the probe.

5 The probe of clause 1 or of any of the preceding clauses, wherein the thermal shield comprises a thermally insulative material.

6. The probe of clause 5, wherein the thermally insulative material is at least one of plastic, glass, and polyurethane.

7. The probe of clause 1 or of any of the preceding clauses, wherein the thermal shield comprises a distal end cap connectable to the distal end of the probe.

8. The probe of clause 7, wherein the distal end cap includes a lock that connects the distal end cap to the probe.

9. The probe of clause 8, wherein the lock comprises at least one of a mechanical lock, a fixed lock, a friction lock, and a thermal lock.

10. The probe of clause 9, wherein the aperture is disposed on a distal tip of the distal end cap.

11. A system, comprising:
a probe comprising:
   a distal electrode disposed on a distal end of the probe; and
   a proximal electrode disposed proximally from the distal electrode; and
a thermal shielding disposable on the probe and configured to selectively thermally insulate at least one of the distal electrode and the proximal electrode, the thermal shielding comprising:
   an aperture through which thermal energy from at least one of the distal electrode and the proximal electrode can be dissipated.

12. The system of clause 11, wherein a position of the aperture relative to the at least one of the distal electrode and the proximal electrode is adjustable to change an amount of thermal energy dissipated from the probe.

13. The system of clause 12, wherein the thermal shielding is rotatable relative to at least one of the distal electrode and the proximal electrode.

14. The system of clause 11 or of any of clauses 11-13, wherein the probe further comprises:

a thermally insulative element configured to translate across the aperture to adjust an amount of thermal energy dissipated from the probe.

15. The system of clause 11 or of any of clauses 11-14, wherein the thermal shielding comprises a thermally insulative material.

16. The system of clause 15, wherein the thermally insulative material is at least one of plastic, glass, and polyurethane.

17. The system of clause 11 or of any of clauses 11-16, wherein the thermal shielding comprises a distal end cap connectable to the distal end of the probe.

18. The system of clause 17, wherein the distal end cap includes at least one of a mechanical lock, a fixed lock, a friction lock, and a thermal lock that connects the distal end cap to the probe.

19. The system of clause 18, wherein the aperture is disposed on a distal tip of the distal end cap.

20. A system, comprising:
a cooling apparatus including an input channel and an output channel;
a probe comprising:
   an active electrode disposed on a distal end of the probe; and
   a return electrode disposed proximally from the active electrode; and
a thermal sleeve disposable over the probe to selectively thermally insulate at least one of the active electrode and the return electrode, the thermal sleeve including an aperture that enables dissipation of thermal energy from the active electrode through the aperture.

1-15. (canceled)

16. A probe, comprising:
a distal electrode disposed on a distal end of the probe;
a proximal electrode disposed proximally from the distal electrode; and
a thermal shield disposable on the distal end of the probe to selectively thermally insulate at least one of the distal electrode and the proximal electrode, the thermal shield including an aperture that enables dissipation of thermal energy from at least one of the distal electrode and the proximal electrode through the aperture.

17. The probe of claim 16, wherein a position of the aperture relative to the at least one of the distal electrode and the proximal electrode is adjustable to change an amount of thermal energy dissipated from the probe.

18. The probe of claim 17, wherein the thermal shield is rotatable relative to at least one of the distal electrode and the proximal electrode.

19. The probe of claim 16, wherein the probe further comprises:
a thermally insulative element configured to translate across the aperture to adjust an amount of thermal energy dissipated from the probe.

20. The probe of claim 16, wherein the thermal shield comprises a thermally insulative material.

21. The probe of claim 20, wherein the thermally insulative material is at least one of plastic, glass, and polyurethane.

22. The probe of claim 16, wherein the thermal shield comprises a distal end cap connectable to the distal end of the probe.

23. The probe of claim 22, wherein the distal end cap includes a lock that connects the distal end cap to the probe.

24. The probe of claim 23, wherein the lock comprises at least one of a mechanical lock, a fixed lock, a friction lock, and a thermal lock.

25. The probe of claim 24, wherein the aperture is disposed on a distal tip of the distal end cap.

26. A system, comprising:
a probe comprising:
a distal electrode disposed on a distal end of the probe; and
a proximal electrode disposed proximally from the distal electrode; and
a thermal shielding disposable on the probe and configured to selectively thermally insulate at least one of the distal electrode and the proximal electrode, the thermal shielding comprising:
an aperture through which thermal energy from at least one of the distal electrode and the proximal electrode can be dissipated.

27. The system of claim 26, wherein a position of the aperture relative to the at least one of the distal electrode and the proximal electrode is adjustable to change an amount of thermal energy dissipated from the probe.

28. The system of claim 27, wherein the thermal shielding is rotatable relative to at least one of the distal electrode and the proximal electrode.

29. The system of claim 26, wherein the probe further comprises:
a thermally insulative element configured to translate across the aperture to adjust an amount of thermal energy dissipated from the probe.

30. The system of claim 26, wherein the thermal shielding comprises a thermally insulative material.

31. The system of claim 30, wherein the thermally insulative material is at least one of plastic, glass, and polyurethane.

32. The system of claim 26, wherein the thermal shielding comprises a distal end cap connectable to the distal end of the probe.

33. The system of claim 32, wherein the distal end cap includes at least one of a mechanical lock, a fixed lock, a friction lock, and a thermal lock that connects the distal end cap to the probe.

34. The system of claim 33, wherein the aperture is disposed on a distal tip of the distal end cap.

35. A system, comprising:
a cooling apparatus including an input channel and an output channel;
a probe comprising:
an active electrode disposed on a distal end of the probe; and
a return electrode disposed proximally from the active electrode; and
a thermal sleeve disposable over the probe to selectively thermally insulate at least one of the active electrode and the return electrode, the thermal sleeve including an aperture that enables dissipation of thermal energy from the active electrode through the aperture.
